(19) United States
(12) Patent Application Publication
(10) Pub. No.: US 2013/0024954 A1
(43) Pub. Date: Jan. 24, 2013

(54) Human Age-Related Neurodegenerative Nematode Model and Methods

(71) Applicants: Jonathan Thomas Pierce-Shimomura, Austin, TX (US); Ashley Crisp, Austin, TX (US)

(72) Inventors: Jonathan Thomas Pierce-Shimomura, Austin, TX (US); Ashley Crisp, Austin, TX (US)

(21) Appl. No.: 13/532,539

(22) Filed: Jun. 25, 2012

Related U.S. Application Data
(60) Provisional application No. 61/500,715, filed on Jun. 24, 2011.

(51) Int. Cl. A01K 67/033; G01N 33/48

(52) U.S. Cl. 800/3; 800/13

(57) ABSTRACT

Genetically modified nematodes and methods for using the same are provided.

### CROSS REFERENCE TO RELATED APPLICATIONS

This application claims priority to U.S. Provisional Application No. 61/500,715, filed Jun. 24, 2011, which is incorporated herein by reference.

### BACKGROUND

Neurodegeneration is the umbrella term for the progressive loss of structure or function of neurons, including death of neurons. Neurodegenerative diseases are usually characterized by onset in late adulthood, a slowly progressive clinical course and neuronal loss with regional specificity in the central nervous system. Many neurodegenerative diseases including Parkinson's, Alzheimer's, and Huntington's occur as a result of neurodegenerative processes. For example, Alzheimer's alone now represents the 6th leading cause of death. These enormous problems demand the development of novel strategies to study and prevent the pathological and “natural” decline of nervous system function with age and age-related neurological diseases.

### SUMMARY

The present disclosure generally relates to degenerative diseases. More particularly, the present disclosure relates to organisms for modeling neurodegenerative diseases and methods for determining the cellular and molecular basis for how neurons degenerate.

According to certain embodiments, the present disclosure provides a genetically modified nematode belonging to genus Caenorhabditis comprising a single additional copy of a gene that encodes an ortholog of a gene associated with a neurodegenerative disease.

According to certain embodiments, the present disclosure provides a genetically modified nematode belonging to genus Caenorhabditis comprising a single copy of at least a portion of a human gene associated with a neurodegenerative disease.

According to certain embodiments, the present disclosure provides a genetically modified nematode belonging to genus Caenorhabditis comprising one or more of the following genes under the control of the egl-l promoter: egl-1, ced-4, ced-3, ced-1 and ced-6.

According to certain embodiments, the present disclosure provides a method comprising exposing a genetically modified nematode according to the present disclosure to a substance; and observing a phenotypic change in the nematode.

According to certain embodiments, the present disclosure provides a method comprising exposing a genetically modified nematode to a substance; observing the effect of the substance on a phenotype of the nematode; and comparing the effect on the observed phenotype in the presence of the substance to the observed phenotype in the absence of the substance, wherein the substance is identified as a pharmaceutical for the treatment or prevention of a neurodegenerative disease.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 are micrographs showing defects in development lead to premature death for worms that overexpress pan-neuronal apl-1. Prab3::apl-1::unc-54UTR or Prab3::huAPP695::unc-54UTR construct integrated as a single cop (SC) or in multiple copies as extrachromosomal array (MC).

FIG. 2 are micrographs showing defects in development lead to premature death for worms that overexpress human APP (huAPP695). Prab3::apl-1::unc-54UTR or Prab3::huAPP695::unc-54UTR construct integrated as a single cop (SC) or in multiple copies as extrachromosomal array (MC).

FIG. 3 is a graph showing defects in development lead to premature death for worms that overexpress pan-neuronal apl-1 and human APP (huAPP695). Prab3::apl-1::unc-54UTR or Prab3::huAPP695::unc-54UTR construct integrated as a single cop (SC) or in multiple copies as extrachromosomal array (MC).

FIG. 4 is a graph showing defects in development lead to premature death for worms that overexpress pan-neuronal apl-1 and human APP (huAPP695). Prab3::apl-1::unc-54UTR or Prab3::huAPP695::unc-54UTR construct integrated as a single cop (SC) or in multiple copies as extrachromosomal array (MC).

FIG. 5 are fluorescent microcopy images showing overexpressed APL-1 is observed in VC neurons, with higher protein enrichment specifically in the somata of VC4 and VC5.

FIG. 6 are micrographs showing worms overexpressing a single copy (SC) of apl-1 or huAPP695 retain more eggs in the gonad as they age compared to wild type. This is exaggerated in worms overexpressing multiple gene copies (MC).

FIG. 7 is a graph showing worms overexpressing a single copy (SC) of apl-1 or huAPP695 retain more eggs in the gonad as they age compared to wild type. This is exaggerated in worms overexpressing multiple gene copies (MC). * Significant compared to age-matched wildtype.

FIG. 8 are micrographs and a graph showing worms overexpressing a single copy of apl-1 show an age-related deficit in swimming, which is exaggerated in MC overexpressing worms. The swimming deficit is reproduced in wild-type worms that have VC4 & VC5 ablated.

FIG. 9 are micrographs showing cholinergic neurons VC4 & VC5 show strong GFP intensity on day 1 of adulthood but become undetectable in 40% of animals by day 3 of adulthood.

FIG. 10 are micrographs showing cholinergic neurons VC4 & VC5 show strong GFP intensity on day 1 of adulthood but become undetectable in 40% of animals by day 3 of adulthood.

FIG. 11 are graphs showing cholinergic neurons VC4 & VC5 show strong GFP intensity on day 1 of adulthood but become undetectable in 40% of animals by day 3 of adulthood.

FIG. 12 are micrographs showing the egg-laying muscles receive synaptic input exclusively from 2 HSN and 6 VC motor neurons. HSNs direct synaptic output to VC5 and onto vulval muscles. VC4 and VC5 direct output to the vulval and ventral body muscles in addition to other VCs.

FIG. 13 are micrographs showing the egg-laying muscles receive synaptic input exclusively from 2 HSN and 6 VC motor neurons. HSNs direct synaptic output to VC5 and onto vulval muscles. VC4 and VC5 direct output to the vulval and ventral body muscles in addition to other VCs.

FIG. 14 are micrographs showing loss of cat-1 alleviated both egg-retention and swimming deficits observed in SC_apl-1 worms. cat-1 encodes VMAT, which is required to package serotonin(5-HT) into vesicles for release.

FIG. 15 is a graph showing loss of cat-1 alleviated both egg-retention and swimming deficits observed in SC_apl-1 worms. cat-1 encodes VMAT, which is required to package serotonin(5-HT) into vesicles for release.

FIG. 16 is a diagram showing loss of caspase egl-1 or engulfment gene ced-6 prevent degeneration of VC4 & VC5.

FIG. 17 is a graph showing loss of caspase egl-1 or engulfment gene ced-6 prevent degeneration of VC4 & VC5.

FIG. 18 is a graph showing that in addition to being cholinergic, VC4 and VC5 neurons are serotonergic and receive 5HT input from HSN neurons. Loss of VMAT, tph-1, or ser-5 alleviated both behavioral deficits and neurodegeneration observed in SC_apl-1 worms.

FIG. 19 is a graph showing drugs that hinder or block 5HT signaling prevent the death of neurons VC4 and VC5. Pharmacological promotion of 5HT signaling does not prevent, and can increase, degeneration of VC4 and VC5.

FIG. 20 shows APP induces age-related degeneration of a specific subset of cholinergic neurons in C. elegans. (A) Time course to middle age. (B) All six VC neurons visualized with GFP with animal outlined in pink. (C) Same individuals on adult days 1 and 3. VC4&5 neurons selectively degenerate in a strain that expresses a single copy of APP (SC_APP). Remnant GFP, green arrows. Cartoon depictions for day 3 below. (d) Quantification of degeneration of different neuronal classes. For statistical comparisons of ratio degeneration, n>124 neurons (62 animals) per bar, planned X2 tests vs expected ratio from same age WT where *, P<0.0001. Error bars, s.e.m.

FIG. 21 APP induces age-related decline of behaviors that depend on specific cholinergic neurons that degenerate. (A,B) Overexpression strains retain significantly more eggs in middle age compared to WT. (C) Normarski photomicrographs of eggs (yellow arrows) retained in the midbody of adults. Arrow, vacuole indicative of neurodegeneration; double arrow, vulva. Inset shows magnified view. Quantification of egg retention (D) and midbody curvature time course (E). Deficits are recapitulated with laser ablation of VC4&5 neurons. For statistical comparisons of egg retention, n>48 animals per bar, planned t-tests vs same age WT (or ablated vs sham) where **, P<0.001 and *, P<0.05. Error bars, s.e.m.

FIG. 22 Pan-neuronally expressed APP accumulates in specific cholinergic neurons that degenerate in middle age. (A,B) Confocal stack images of mCherry-tagged APL-1 and APP localization in the midbody and head of SC_apl-1 (A) and SC_APP (B) strains. mCherry-tagged protein co-localizes in VC4&5 neurons starting on first day of adulthood but is undetectable in other areas including head. Brackets, area with head neurons that lacks noticeable mCherry signal; asterisks, gut autoflourescence; scale bars, 40 mm.

FIG. 23 APP induces patterned neurodegeneration via an apoptotic pathway that requires egl-1, ced-3, and ced-6. (A) Quantification of VC4&5 degeneration in cell-death pathway mutants. For statistical comparisons of ratio degeneration, n>124 neurons (62 animals) per bar, planned X2 tests vs expected ratio from same age SC_apl-1 where *, P<0.0001. n.s., no significant difference. (B,C) Null mutation in egl-1 preserved WT-like egg-laying and swimming behaviors in SC_APP:mCh strain. For statistical comparisons of egg retention, n>48 animals per bar, planned t-tests vs same age WT where **, P<0.05. Error bars, s.e.m. (D) mCherry-tagged APP still accumulates in VC neurons spared from degeneration in egl-1(lf) background, inset. Asterisks, gut autoflourescence; scale bars, 40 μm.

FIG. 24 Distinct portions of APP are sufficient to induce degeneration of specific cholinergic neurons. (A) ClustalW alignment shows that the intracellular domain of human APP is highly conserved with the equivalent portion of C. elegans APL-1 protein. (B) Quantification of VC4&5 neurodegeneration (n>124 neurons, 62 animals per bar). Single-copy overexpression of the intracellular domain of APP is sufficient to induce neurodegeneration and behavioral decline, similar to that seen in SC_apl-1 and SC_APP strains. Statistically compared with planned X² tests vs expected ratio from same age WT where *, P<0.0001. (C,D) Behavioral decline in egg retention (n>48 per bar) (C) and swim head-bend frequency (n>20 per bar) (D) in APPC59 worms. For panels C and D, statistically compared with planned t-tests vs same age WT where *, P<0.01.

FIG. 25 P7C3 and Dimebon prevent APP-induced neurodegeneration by blocking entrance to apoptosis. (A) P7C3 and Dimebon both prevent degeneration of VC4&5 neurons. Planned X² tests vs expected ratio from untreated animals of same age and genotype where *, P<0.00001. (B) Dose-response curves. Planned X² tests vs expected ratio from untreated animals of same genotype where *, P<0.001. (C) Protective effects of drugs on neurodegeneration are not additive in an egl-1 (null) background. Drugs cannot prevent degeneration induced by gain-of-function mutation in egl-1. Planned X² tests vs expected ratio from untreated animals of same age and genotype where #, P<0.001. (D) Confocal stack images show mCherry-tagged APP still accumulates in neurons spared from degeneration with treatment of P7C3, inset. Asterisks, gut autoflourescence; scale bars, 40 mm. For panels A-C all bars and data points represent n>124 neurons, 62 animals per bar.

FIG. 26 Quantification of APP and apl-1 expression. A. mRNA expression levels of sequence shared with apl-1 and APPare about 2-fold higher in SC overexpression strains compared to WT. Strains that express multiple copies (MC) of APP or apl-1 show even higher levels of expression. Primers used were common to both APP and apl-1 sequences (see methods). B. MC strains show much higher level of lethality compared to SC strains, which are not statistically different than a control strain (designated WT for this paper) that has a control gene knocked into the same genetic locus. Statistically compared with planned X2 tests vs expected ratio from WT where *, P<0.0001. C. Quantification of VC 4&5 degeneration shows indistinguishable pattern of degeneration for SC_apl-1 and SC_APP strains generated on different chromosomes (II and IV) under control of pan-neuronal promoter (Prab-3) or using endogenous promoter Pap1-1 (n>124 neurons, 62 animals per bar). All day-3 and day-5 data are significantly higher than LX959 expressing GFP in VC neurons (P<0.001). Horizontal lines for comparison with day-3 adult WT and SC_apl-1 data.

FIG. 27 Incidence of degeneration of VC cholinergic neurons. Although all six of the VC-class cholinergic neurons show age-related progression of degeneration, neurons VC4 and VC5 show the highest incidence of degeneration. n>124 neurons, 62 animals per bar.

FIG. 28 APP overexpression produces same effects regardless of chromosomal insertion site and promoter. A,B. Egg-retention defects were similar in SC_apl-1 and SC_APP strains generated on different chromosomes (II or IV) with pan-neural promoter (Prab-3), using the endogenous apl-1 promoter, or after laser-ablation of VC4&5 neurons in a LX959 background (data same as in FIG. 2) (n>48 per bar). Asterisks denote significant difference from WT in (A) and from same condition dead vs alive.(B). C. Deficits in head-bend frequency of SC_apl-1 and SC_APP swimming correlates with VC4&5 death, irrespective of chromosome integration site (n>20 per bar). For all panels, statistically compared with planned t-tests vs same age WT where *, P<0.01, expect for ablated animals which were compared to same age sham animals where #, P<0.05.

FIG. 29 Localization of APP in VC-class cholinergic neurons. A,B. In rare animals that reach advanced age (day 10 adults) mCherry-tagged APL-1 (A) and APP (B) both localize to VC neurons, in addition to surviving VC4&5 neurons, in day 10 animals. Green arrows, location of dead neurons; asterisks, gut autoflourescence; scale bars, 40 m.

FIG. 30 Confirmation that Prab-3 is a pan-neuronal promoter. A. A single copy Prab-3::mCherry knocked into the genome is found expressed in neurons throughout the ventral nerve cord, including VC neurons as indicated by double labeling (yellow) with GFP-specific VC neuron reporter (white arrows). B,C. The pan-neuronal promoter Prab-3 expresses mCherry (displayed white) throughout nervous system in a SC_APP background (B) and also in VC neurons (yellow indicates overlap of red mCherry signal with green VC neurons) (C). D,E. The endogenous promoter region (2 kb) of apl-1 expresses mCherry (displayed white) throughout nervous system in a SC_APP background (d) and also in VC neurons (yellow indicates overlap of red mCherry signal with green VC neurons) (e). Worms are positioned in coiled posture to show entire nervous system and asterisks indicate numerous neurons in head in panels D and E. For panels A-D scale bars, 40 μm.

FIG. 31 P7C3 and Dimebon can prevent degeneration induced by full-length AP and intracellular APP but not extracellular APP. Quantification of VC4&5 neurodegeneration (n>124 neurons, 62 animals per bar). Statistically compared with planned t-tests vs same age and genotype where *, P<0.01.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to degenerative diseases. More particularly, the present disclosure relates to organisms for modeling neurodegenerative diseases and methods for determining the cellular and molecular basis for how neurons degenerate.

Shifting demographics are leading to a historically high proportion of elderly worldwide. This has raised the incidence of individuals with age-related neurological disorders including Alzheimer's disease and Parkinson disease. Unfortunately, there are currently no FDA approved drugs available to prevent or delay neurodegeneration that accompanies these disorders and also in natural aging. The struggle to find drugs that effectively prevent neurodegeneration is complicated by the slow speed and high cost required to test drugs on rodent models of aging and disease. These conventional methods require administration of experimental drugs over 10-months to 2 years until the rodent reaches middle or advanced age and begins to display behavioral evidence of cognitive decline. Furthermore, the rodent brains must then be harvested, sectioned, stained, and to count hundreds of neurons in what are often ambiguous models of human disease.

The present disclosure provides, according to certain embodiments, a genetically modified nematode belonging to genus Caenorhabditis comprising a single additional copy of a gene that is associated with a neurodegenerative disease. The nematode may belong to any species, such as for example, elegans, vulgaris, and briggsae.

In certain embodiments, the gene may be an ortholog of a gene associated with a neurodegenerative disease (e.g., apl-1). In other embodiments, the gene may be at least a portion of a human gene associated with a neurodegenerative disease (e.g., APP). The gene is inserted into the somatic genome as a single copy. Any gene may be suitable so long as it is associated with a neurodegenerative disease. Examples of suitable genes include, but are not limited to, genes associated with Alzheimer's disease (AD), Parkinson's disease (PD), spinocerebellar ataxias (SA), amyotrophic lateral sclerosis (ALS), Schizophrenia, Huntington's disease (HD), Down Syndrome (DS), and natural aging.

TABLE 1

| | Nematode | Human |
|---|---|---|
| Disease | ortholog | gene |
| Alzheimer's disease (AD) | apl-1 | APP |
| | ptl-1 | TAU |
| | Y110A2Al.13 | PIN1 |
| Parkinson's disease (PD) | pink-1 | PINK |
| spinocerebellar ataxias (SA) | K04F10.1 | SCA1 |
| | atx-2 | ataxin- 2 |
| | atx-3 | ataxin-3 |
| | abtm-1 | ABCB7 |
| Down Syndrome (DS) | unc-23 | SYNJ1 |
| | irk-1, irk-2 | IRK3 |
| | ZK1320.13 | PCP4 |

In certain embodiments, the gene may comprise a tag, which may be useful for visualizing the expression of the gene. Examples of suitable tags include, but are not limited to, fluorescent tags, such as fluorescent reporter genes (e.g., enhanced green fluorescent protein (EGFP), tdTomato, YFP, CFP, and mCherry), and epitope tags for antibody labeling (e.g. HA and FLAG).

In certain embodiments, the genetically modified nematode may display a phenotype associated with the expression of the gene. Such phenotypes may be characteristic of a neurodegenerative disease or a lack thereof. Examples of phenotypes that may be associated with the expression of the gene include, but are not limited to, a defect in egg laying; a defect in swimming and/or crawling; a defect in defecation; a defect in molting; a defect in the formation of the vulva; plaque-like deposits in the nervous system; a retarded development; a decreased number of descendants; fluorescence; fragmentation of neural processes; condensation of neural somata; and growth of aberrant neural processes. The phenotype, or change in phenotype, may be used in methods for identifying suitable therapeutic candidates, according to certain embodiments.

By way of explanation, and not of limitation, insertion of only a single copy of the gene is thought to avoid sever phenotypes associated with high gene dosages. The genetically modified nematodes of the present disclosure may display a very low level of lethality as compared to control stains with a single-copy of a control transgene knocked into the same locus. Moreover, genetically modified nematodes of the present disclosure that escape embryonic lethality may appear normal in morphology and development from egg to young adult, and may display an expected 2-fold higher expression of the gene mRNA transcript than wild-type. Thus, the nematode models of the present disclosure may better mimic the protein load found in human patients with a neurodegenerative disease.

The present disclosure provides, according to certain embodiments, a genetically modified nematode belonging to genus Caenorhabditis comprising one or more of the following genes under the control of the egl-l promoter: akt-1, gld-1, ape-1, abl-1, fsn-1, egl-1, egl-1, ced-4, ced-3, ced-1 and ced-6. The genes may be present in the genetically modified nematode in single- and/or multi-copy.

The nematodes of the present disclosure may be used to observe the neurodegeneration of small subsets of neurons. Such observations may be performed directly and non-invasively through the animal's transparent body for easy quantification. In some of the models, the entire morphological integrity of each neuron is more easily monitored by using neuron class-specific expression of a fluorescent reporter gene (e.g., enhanced green fluorescent protein (EGFP) or mCherry). Furthermore, the function of the neurons in question can be assessed by quantitatively monitoring discrete behaviors that depend on those neurons. Observation of the nematodes and quantification of neurodegeneration may be automated.

Accordingly, the present disclosure also provides, according to certain embodiments, methods for screening substances that prevent and/or delay neurodegeneration using the genetically modified nematodes of the present disclosure. In general, such methods comprise exposing a genetically modified nematode to a substance and observing a phenotypic change in the nematode. For example, in certain embodiments a method may comprise culturing a genetically modified nematode on agar-filled plates that contain a chemical compound and the morphological integrity and life/death status of neurons in treated nematodes are viewed directly over the course of the animal's life (for example, using low- and/or high-power microscopy and/or DIC and fluorescence microscopy).

While age-related neurodegeneration can take months to years in other mammalian models of neurodegeneration, the short life cycle of nematodes allows for neurodegenerative progression over a few days. Moreover, the nematode models of the present disclosure allow for testing the functional status of specific neurons by monitoring behaviors (i.e., phenotypes) of drug-treated nematodes. Additionally, in certain embodiments, the methods may be configured for high-throughput screening and/or automation.

The methods of the present disclosure may be used to identify substances that may be useful for treating human neurological disorders. In certain embodiments, methods of the present disclosure include determining a subset of compounds that prevent and/or delay neurodegeneration in genetically modified nematodes of the present disclosure. In other embodiments, methods of the present disclosure include testing which points in the cell-death pathway a set of compounds might act to prevent or delay neurodegeneration. Such methods may be used to elucidate which point in a molecule pathway a particular compound acts to prevent neurodegeneration. The neurodegenerative process in most neurological diseases is hypothesized to occur through the activation of key genes in “cell death” pathways such as apoptosis and necrosis. Most all of these genes are conserved between humans, rodents, and C. elegans. Accordingly, in certain embodiments, the present disclosure provides genetically modified nematodes that over express genes that activate cell death in a key set of neurons involved in egg laying behavior. Wild-type worms have no trouble laying up to 10 eggs per hour. These genetically modified nematodes fail to lay eggs during early adulthood and accumulate them until the eggs hatch inside their bodies (“bag-of-worms” phenotype). These genetically modified nematodes may be used to test compounds for their ability to prevent cell death in these strains. Evidence of the ability to prevent and/or delay degeneration will be apparent because ten worms will lay hundreds of eggs on an agar plate impregnated with the compound. Moreover, fewer adult worms will form “bag-of-worms” which are readily visible as immobile bloated animals with writhing babies trapped inside. The set of worms will have the following C. elegans genes over-expressed under the control of the egl-l promoter: akt-1, gld-1, ape-1, abl-1, fsn-1, egl-1, egl-1, ced-4, ced-3, ced-1 and ced-6. Another set of worms overexpress the human gene equivalents: BH3, APAF-1, CASPASE-9, Lrp, and Gulp.

For example, a compound suspected of blocking cell death may be studied to determine how general the effect and what the relevant in vivo targets is in the cell death pathway. Genetically modified nematodes belonging to genus Caenorhabditis comprising one or more cell death pathway genes may be exposed to a substance to determine which subset of strains the compound is effective in preventing degeneration. If the compound can prevent cell death in, for example, egl-1, but not ced-9 and ced-3, then we can conclude that the drug acts downstream of the egl-1, but before ced-9, in the cell death pathway.

To facilitate a better understanding of the present invention, the following examples of certain aspects of some embodiments are given. In no way should the following examples be read to limit, or define, the scope of the invention.

### EXAMPLES

### Example 1

Previous work has pioneered the use of the powerful model organism Caenorhabditis elegans to study the natural and pathological functions of APP.^{4,5} Most likely due to gene dosage effects, current C. elegans models using overexpression of the APP ortholog apl-1 have made it impossible to observe potential neurodegeneration and determine whether defects in behavior reflect neural dysfunction or gross defects in development. Recent advances have now made single-copy overexpression techniques possible⁶, allowing a more physiologically relevant AD model to be made in this study. The simple nervous system, rapid lifecycle, and ease of genetic manipulation make it possible to track individual neurons over time in C. elegans and to characterize age-related effects. Our AD model will also lend itself to exploration of cellular mechanisms by which APP or its ortholog lead to disease pathology.

Previous attempts to produce a model of AD in C. elegans have been less productive, in large part due to the extreme phenotypes that result from the overexpression of multiple copies of APP. For example, in contrast to the mouse model, multi-copy overexpression of the APP ortholog, apl-1, led to a collection of extreme phenotypes including partial lethality, arrested development, and extensive vacuolization. These findings were also confirmed by overexpressing multiple copies of the apl-1 gene and the APP gene with a pan-neuronal promoter. Neurodegeneration could not be easily discerned in these animals. Utilizing a new technique⁶, which allows the insertion of a single gene into the C. elegans genome, it is now possible to determine if an extra single copy of the apl-1 or APP results in phenotypes that better reflect the human AD condition.

We have now discovered that, as observed in human AD, overexpression of a single wild-type copy of the APP ortholog results in age-related degeneration of a subset of cholinergic neurons in C. elegans. The time course of degeneration can be followed noninvasively through the animal's transparent body with DIC and fluorescence microscopy. To quantify the impact of degeneration, we have devised image analysis algorithms to track the deterioration of two natural behaviors (swimming and egg laying) that depend on these neurons. The quantifiable link between these behaviors and neurodegeneration allows us a direct approach for accessing strategies to recover the function mediated by the degenerated cholinergic neurons.

Here we present the first example of APP-induced degeneration of cholinergic neurons in C. elegans.

Methods and Results

Using C. elegans, we investigated how overexpression of human APP and its worm ortholog, apl-1, lead to the degeneration of specific neuron types, including ACh and 5HT.

Manipulations of C. elegans worms may be performed using techniques known in the art, including those described in Methods in Cell Biology, vol 84; Caenorhabditis elegans: modern biological analysis of an organism, ed. Epstein and Shakes, academic press, 1995, or using minor modifications of the methods described therein.

Generate Single-Copy Overexpression Worms Using Recombination Techniques and Microinjection of apl-1 and APP.

Transgenic animals with a single copy of the apl-1 transgene are generated through the technique summarized in Frokjaer-Jensen C, Davis M W, Hopkins C E, Newman B J, Thummel J M, Olesen S P, Grunnet M, Jorgensen E M. 2008. Single-copy insertion of transgenes in Caenorhabditis elegans. Nat. Genet. 40: 1375-83. First, Gateway technology (Invitrogen) is used to construct a vector with the apl-1 gene driven by a pan-neuronal (Prab-3) promoter. The apl-1 transgene is then linked to the wild-type unc-119 gene. The unc-119 transgene is used for positive selection when injected into unc-119 uncoordinated mutant animals. Utilizing the MosSCI technique, DNA complementary to a Mos1 transposon insertion site on the 2^{nd} chromosome flanks the apl-1 and unc-119 gene. Worms are injected with this vector and a transposase vector to trigger mobilization of the Mos1 transposon. Full insertion of apl-1 is then confirmed in motile progeny by PCR and sequencing across the transposon insertion site. The relative level of copy number will be determined using QPCR. This single-copy transgenic strain is termed SC_apl-1. The process will be repeated to generate a strain that overexpresses one copy of huAPP695, the human allele most abundant in the nervous system (SC_APP). A strain that only has a single copy of the unc-119 gene inserted will act as a control for these overexpression strains (SC_con).

Characterize and Quantify Deficits in Gross Body Morphology or Specific Natural Behaviors in Overexpression Worms.

Worms generated according to the above procedure are immobilized on 10% agarose pads without anesthetic and photographed at 40× magnification while being visually inspected for any apparent morphological defects. To quantify any deficits in locomotion, individual worms are video recorded for 60 seconds while crawling on an agar plate and swimming in water. High-throughput tracking software was developed and used to track the midline of the animal and efficiently quantify the kinematics of both the overexpression worms and non-overexpressing controls. Pierce-Shimomura J T, Chen B L, Mun J J, Ho R, Sarkis R, McIntire S L. 2008. Genetic analysis of crawling and swimming locomotory patterns in C. elegans. Proc Natl Acad Sci USA. 105: 20982-7, incorporated by reference. Computer analysis quantifies subtle phenotypes that might not have been identified qualitatively through observation. To identify any abnormalities in the egg-laying circuit, individual adult worms are dissolved in 5% bleach solution to assess egg retention. Eggs remain intact due to their thick chitin shell. The average number and stage of eggs per adult individual is calculated for both the overexpression strains and non-overexpressing controls, with either elevated or lower numbers indicative of malfunctioning circuitry.

Generate APL-1 and APP Overexpression Strains with Fluorescently Labeled Neuronal Classes.

Many transgenic strains can be easily obtained from a public consortium (CGC), including those with specific neurons fluorescently labeled in a variety of colors. If not publicly available, these strains can be easily constructed using a PCR fusion approach. Final PCR products contain the GFP gene driven by a specific promoter-of-interest and are then microinjected into the gonad of adult wildtype animals. C. elegans have the ability to form a stable, functional extra-chromosomal array with injected PCR products. Therefore, they express injected DNA. Fluorescent worms are then crossed to the single-copy overexpression worms to generate overexpression worms with different identifiable sets of neurons labeled.

Protein-tagged constructs are used to track location of APL-1 or APP protein produced within the worm. Generation of these worms is identical to that outlined above, with the exception of having the fluorescent mCherry gene sequence just upstream of the 3′ untranslated region. Once translated, the C-terminal portion of the protein will be tagged with mCherry, which can be visualized under fluorescent microscopy. Upon protein cleavage, the C-terminal end of APL-1 and APP are released intracellularly and may be responsible for aggregate formation and neuronal death. Worms will be visually assessed each day for 8 days using a confocal microscope. The appearance and changes in fluorescence intensity over time can then be quantified to determine cellular localization and accumulation of APL-1 or APP protein in specific cells.

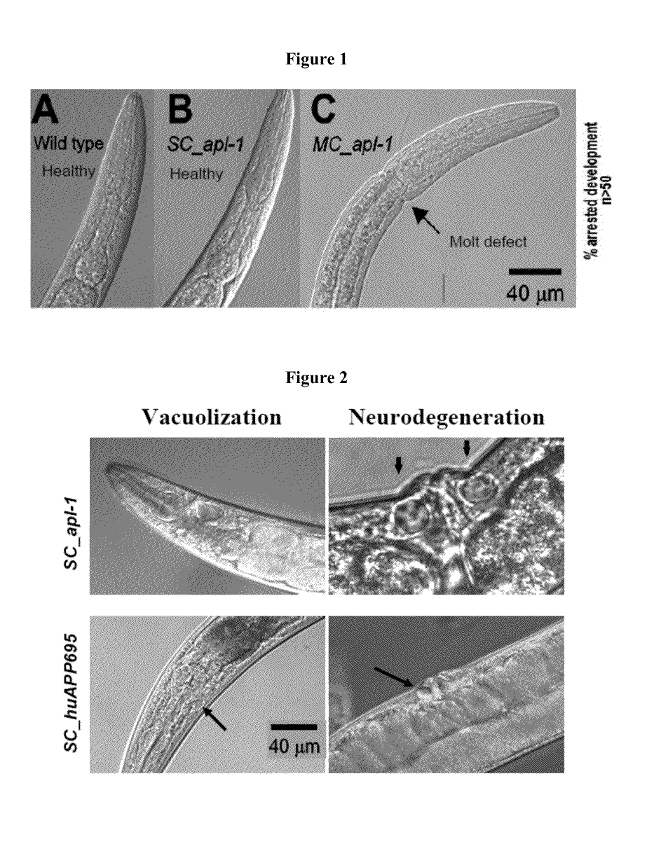

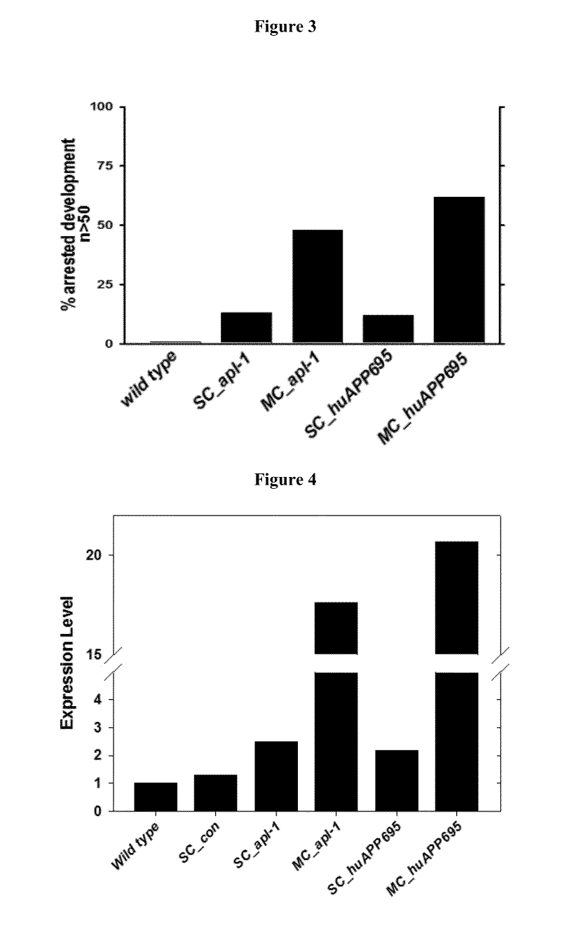

As shown in the FIG. 1, FIG. 2, FIG. 3, and FIG. 4, defects in development lead to premature death for worms that overexpress pan-neuronal apl-1. This is also seen in worms overexpressing human APP (huAPP695).

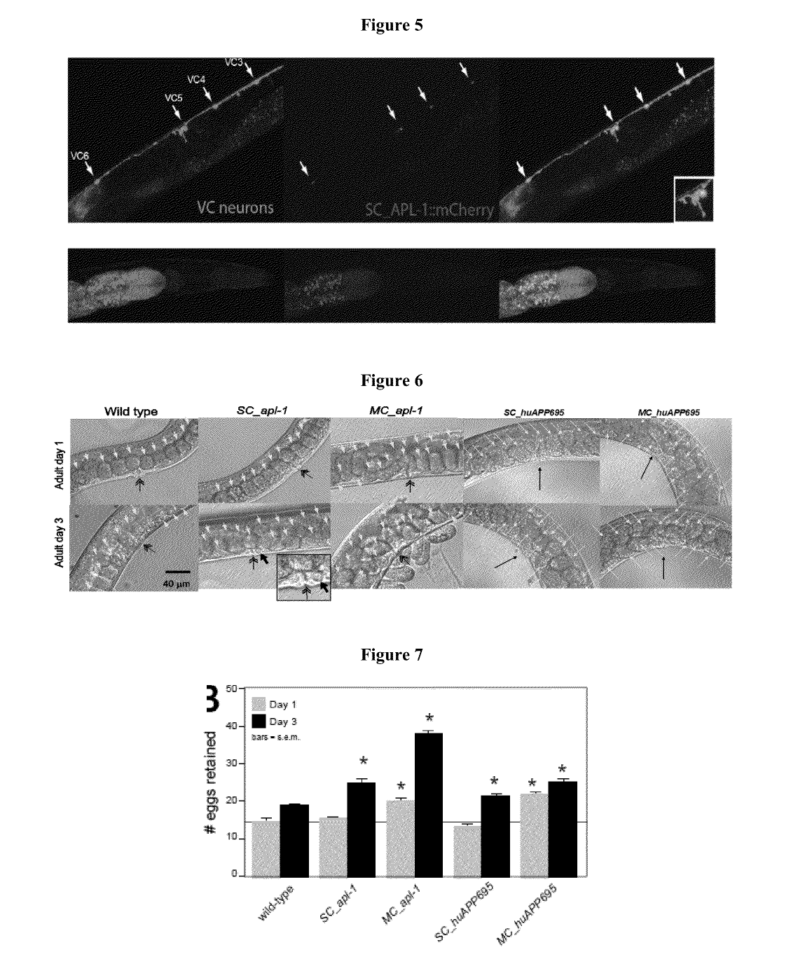

As shown in the FIG. 5, overexpressed APL-1 is observed in VC neurons, with higher protein enrichment specifically in the somata of VC4 and VC5.

As shown in the FIG. 6 and FIG. 7, worms overexpressing a single copy (SC) of apl-1 or huAPP695 retain more eggs in the gonad as they age compared to wild type. This is exaggerated in worms overexpressing multiple gene copies (MC).

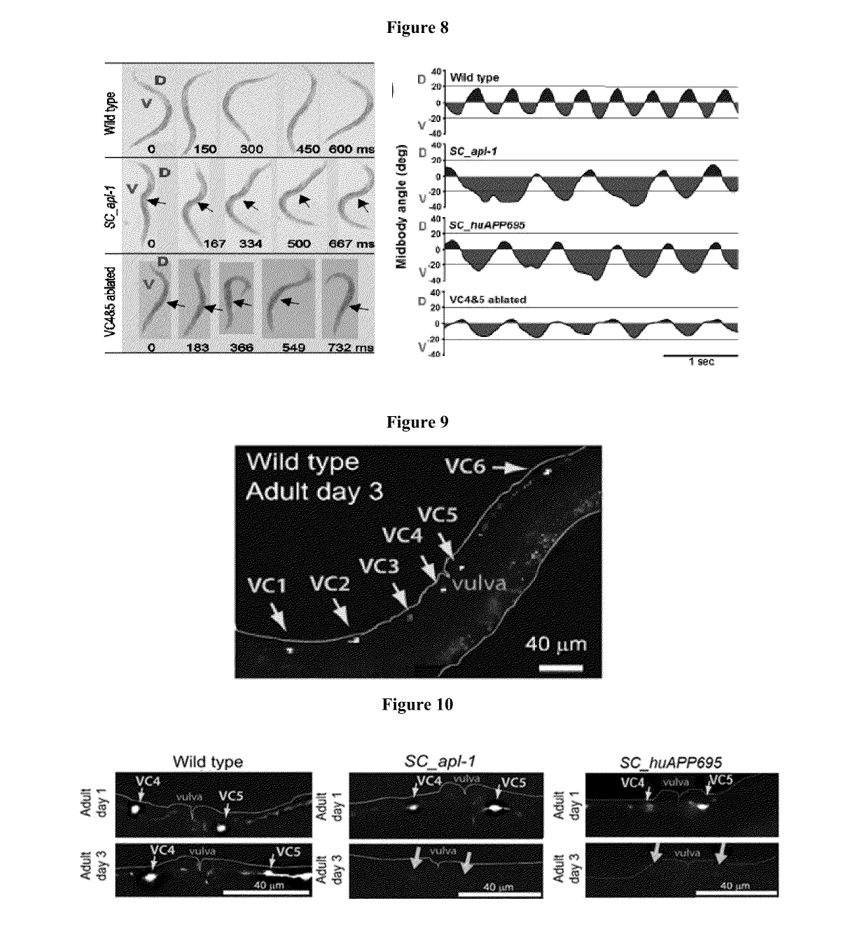

As shown in FIG. 8, worms overexpressing a single copy of apl-1 show an age-related deficit in swimming, which is exaggerated in MC overexpressing worms. The swimming deficit is reproduced in wild-type worms that have VC4 & VC5 ablated.

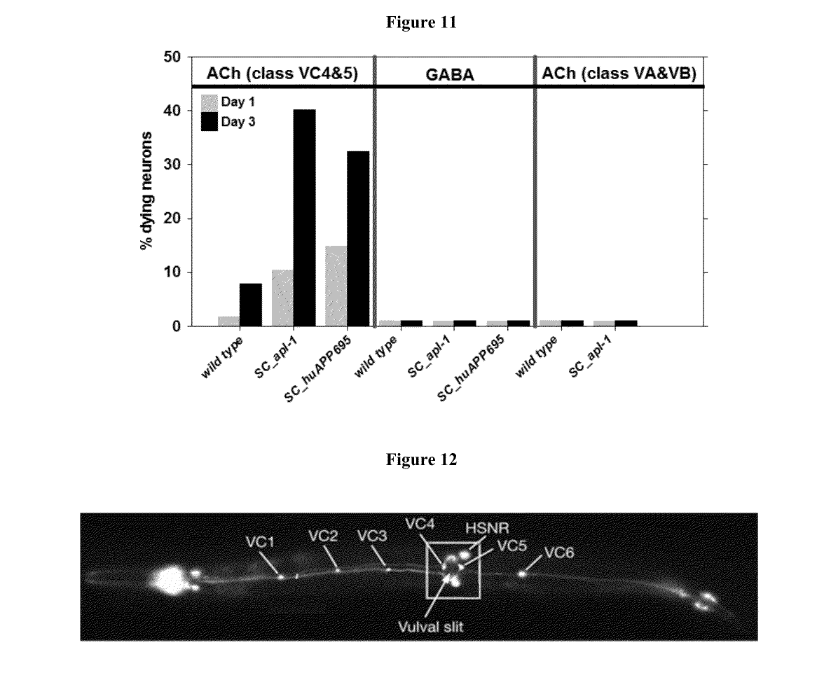

As shown in the FIG. 9, FIG. 10, and FIG. 11 cholinergic neurons VC4 & VC5 show strong GFP intensity on day 1 of adulthood but become undetectable in 40% of animals by day 3 of adulthood.

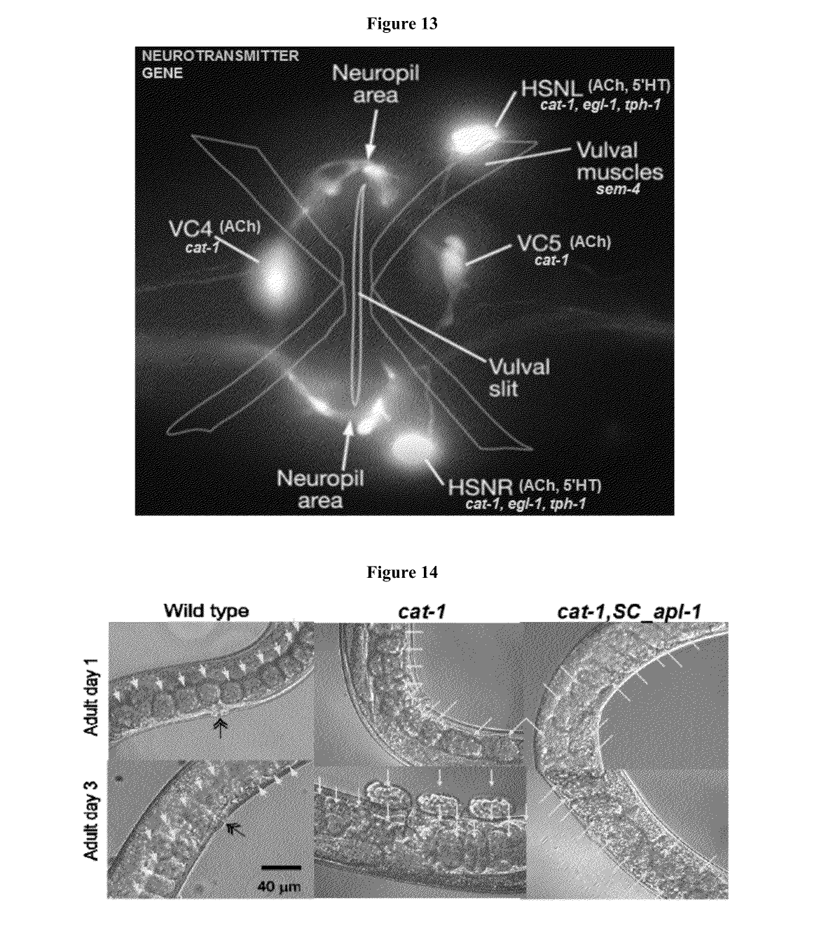

The egg-laying muscles receive synaptic input exclusively from 2 HSN and 6 VC motor neurons. HSNs direct synaptic output to VC5 and onto vulval muscles. VC4 and VC5 direct output to the vulval and ventral body muscles in addition to other VCs. (FIG. 12 and FIG. 13.)

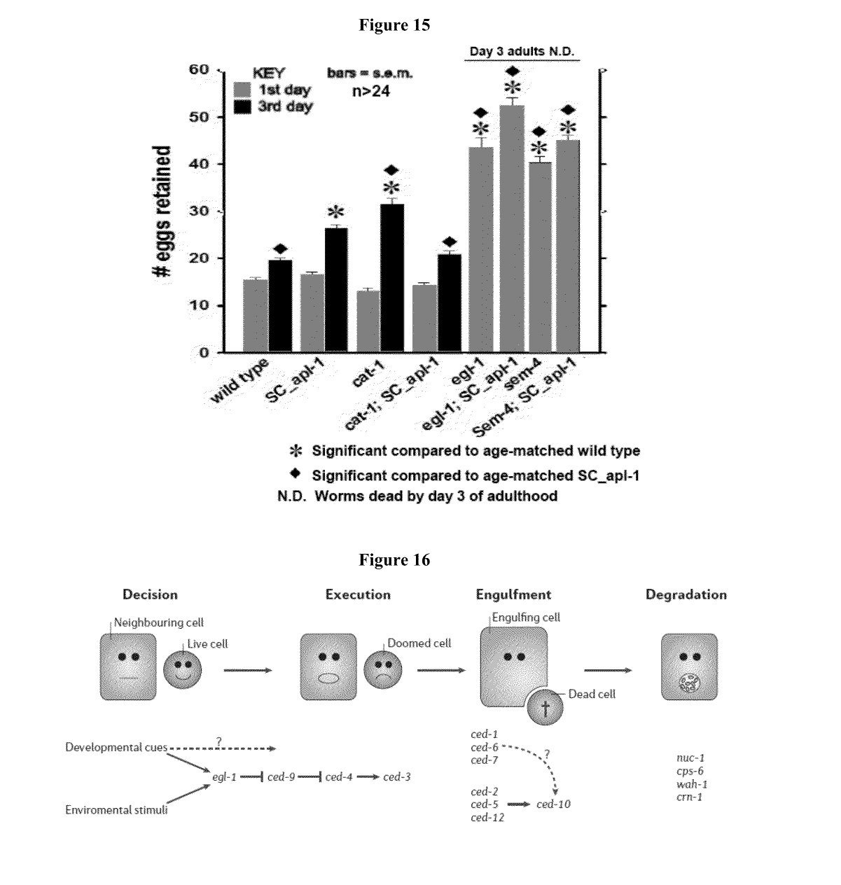

As shown in the FIG. 14 and FIG. 15, loss of cat-1 alleviated both egg-retention and swimming deficits observed in SC_apl-1 worms. cat-1 encodes VMAT, which is required to package serotonin(5-HT) into vesicles for release.

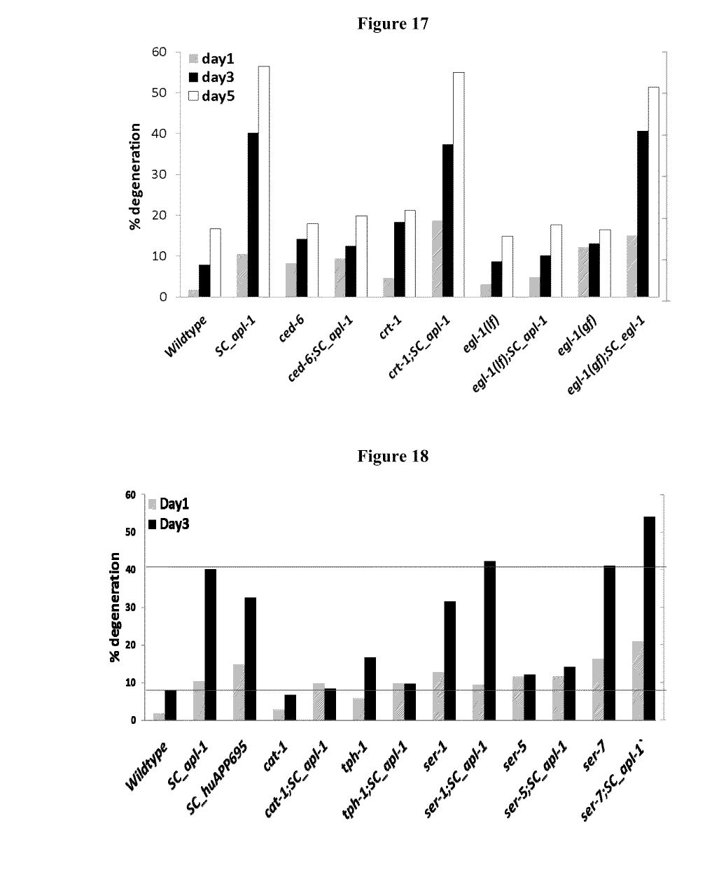

As shown in the FIG. 16 and FIG. 17, loss of caspase egl-1 or engulfment gene ced-6 prevent degeneration of VC4 & VC5

As shown in FIG. 18, in addition to being cholinergic, VC4 and VC5 neurons are serotonergic and receive 5HT input from HSN neurons. Loss of VMAT, tph-1, or ser-5 alleviated both behavioral deficits and neurodegeneration observed in SC_apl-1 worms.

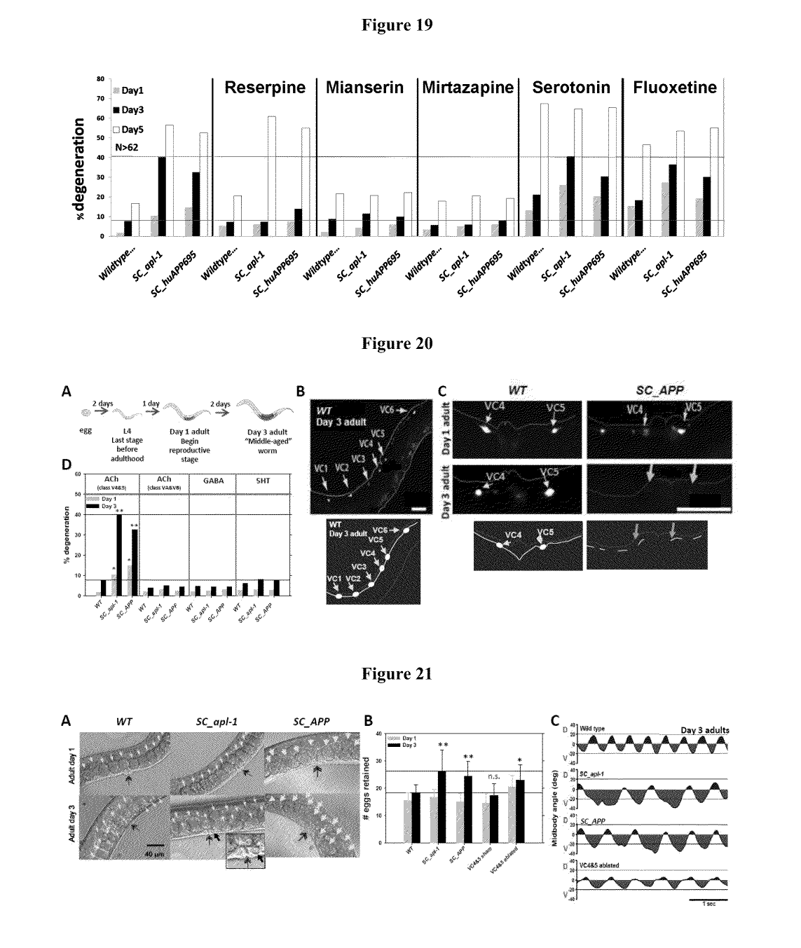

Drugs that hinder or block 5HT signaling prevent the death of neurons VC4 and VC5. Pharmacological promotion of 5HT signaling does not prevent, and can increase, degeneration of VC4 and VC5. (FIG. 19.)

TABLE 2
Drugs acting on 5HT signaling pathway

| Drug | Action |
|---|---|
| Reserpine | Blocks VMAT |
| Mianserin, Mirtazapine, | 5HT receptor antagonists |
| SB299885, SB742757 | |
| Fluoxetine | Selective serotonin reuptake inhibitor |
| Serotonin | 5HT receptor agonist |

Although a similar 5HT model has not yet been implicated in human AD, 5HT has been implicated in influencing the birth and survival of adult-born neurons involved in memory in the hippocampus. The 5HT receptor antagonists that we used (Mianserin, Mirtazapine, SB299885, and SB742757) to prevent APP-induced degeneration are similar in structure to drugs (e.g. Dimebon) that have been found to promote the survival (prevent death) of adult-born hippocampal neurons. All three drugs are also related by having antihistamine properties in humans and sharing certain metabolic products. The mechanism by which Dimebon promotes the survival (prevent death) of adult-born neurons remains unknown. As shown in the table below, we now demonstrate that Dimebon also prevents APP-induced neurodegeneration in C. elegans. Dimebon has been recently used to treat human AD and mouse models of AD. Historically, Dimebon produced the best results for AD out of any drug in clinical trials. Our findings that neurons vulnerable to death in worm, mouse and human are rescued by Dimebon demonstrate that a human AD drug can effectively prevent neurodegeneration in our worm models, and as such, our worm AD models can be used to test for additional drugs that may be effective in preventing or delaying neurodegeneration in AD and other neurodegenerative disorders.

TABLE 3
Dimebon

| Strain | Day 1 | % degen | Day 3 | % degen | Day 5 | % degen |
|---|---|---|---|---|---|---|
| Wild-type | 5 of 108 | 4.63% | 8 of 124 | 6.45% | 15 of 116 | 12.93% |
| SC_apl-1 | 14 of 156 | 8.97% | 27 of 170 | 15.88% | 12 of 68 | 17.64% |
| SC_huAPP695 | 13 of 122 | 10.66% | 17 of 110 | 15.45% | 13 of 78 | 16.67% |

Pan-neuronal overexpression of a single wild-type copy of apl-1 or huAPP695 in C. elegans causes age-related neurodegeneration in a specific subset of cholinergic neurons. Mutation in cat-1 alleviate the deterioration of two natural behaviors that depend on these neurons. Blocking the 5HT system alleviates the deterioration of two natural behaviors that depend on these neurons. Our results are consistent with recent findings from worm and mouse models of AD, in which the serotonin system is preferentially affected by APP overexpression. This pattern of degeneration mimics AD in humans and suggests a functional conservation between the two genes.

### Example 2

### Experimental Procedures

Strains.

C. elegans strains were grown at 20° C. as described in Brenner S. (1974) The genetics of Caenorhabditis elegans. Genetics 77, 71-94. The genotypes of mutant and transgenic strains generated for this study were confirmed through PCR and/or sequencing and are listed with other strains used in Table 4.

TABLE 4
Transgenic strains generated and other mutants.

| Number | Strain & Shorthand | Genotype |
|---|---|---|
| 1 | JPS6 | vxSi1[Prab3::apl-1::unc-54UTR, Cb-unc-119 (+)] II; |
| | SC_apl-1 | unc-119(ed3) III. |
| 2 | JPS7 | vxEx1[Prab3::apl-1::unc-54UTR, Cb-unc-119 (+)]; |
| | MC_apl-1 | unc-119(ed3) III. |
| 3 | JPS11 | vxSi1 II; vsIs13 IV; lin-15B(n765) X. |
| 4 | JPS22 | vxSi50[Cb-unc-119 (+)] II. |
| | SC_con | (designated WT for this paper) |
| 5 | JPS26 | vsIs13 IV; egl-1(n4065) V; lin-15b(n765) X. |
| 6 | JPS27 | vxSi1 II; unc-119(ed3) III; vsIs13 IV; egl-1(n4065) V; |
| | | lin-15b(n765) X. |
| 7 | JPS35 | vxSi35[Prab3: apl-1: mCherry-unc-54-UTR, Cb-unc- |
| | SC_apl-1: mCh | 119(+)] II; unc-119 (ed3) III. |
| 8 | JPS37 | pha-1(e2123); mdIs162; vxSi1 II; unc-119(ed3) III. |
| 9 | JPS38 | vxSi38[Prab3: huAPP695: unc54UTR, Cb-unc119(+)] II, |
| | SC_APP | unc-119(ed3) III. |
| 10 | JPS39 | vxEx39[Prab3: huAPP695: unc54UTR, Cb-unc-119 (+)], |
| | MC_APP | unc-119(ed3)III. |
| 11 | JPS40 | vxSi35 II; unc-119 III; vsIs13 IV; lin-15B(n765) X. |
| 12 | JPS57 | ced-6(tm1826) III; vsIs 13IV; lin-15B(n765) X. |
| 13 | JPS66 | ced-3(ok734), vsIs13 IV; lin-15B (n765) X. |
| 14 | JPS67 | vsIs38 II; unc-119(ed3) III; vsIs13 IV; lin-15B(n765) X. |
| 15 | JPS70 | vsIs13 IV; crt-1(ok948), lin-15B(n765) V. |
| 16 | JPS71 | vsSi 1 II; ced-6(tm1826), unc-119(ed3) III; vsIs 13IV; |
| | | lin-15B(n765) X. |
| 17 | JPS75 | vxSi1 II; unc-119(ed3) III; vsIs13 IV; crt-1(ok948), |
| | | lin-15B(n765) V. |
| 18 | JPS98 | vsIs13 IV; lin-15B(n765) X; vxEx98[Prab-3::mCherry, |
| | | Cbunc-119 (+)]. |
| 19 | JPS112 | unc-119(ed3) III; vxSi38 IV. |
| | SC_APP (IV) | |
| 20 | JPS113 | unc-119(ed3) III; vsIs13, vxSi1 IV; lin-15b(n765) X. |
| 21 | JPS114 | vxSi II; unc-119(ed3) III; vs48[unc-17::GFP] |
| 22 | JPS115 | vsIs13 IV; egl-1(ok1418) V; lin-15b(n765) X. |
| 23 | JPS116 | vxSi1 II; unc-119(ed3) III; vsIs13 IV; egl-1(ok1418) V; |
| | | lin-15b(n765) X. |
| 24 | JPS126 | unc-119(ed3) III; vxSi1 IV. |
| | SC_apl-1 (IV) | |
| 25 | JPS127 | unc-119(ed3) III; vsIs13, vxSi38 IV; lin-15b(n765) X. |
| 26 | JPS128 | VxSi128[Prab3::huAPP695::mcherry: unc54UTR, |
| | SC_APP: mCh | Cbunc-119(+)] II; unc-119(ed3) III; vsIs13 IV; |
| | | lin-15b(n765) X. |
| 27 | JPS145 | vxSi [Papl-1: apl-1: unc54UTR, Cbunc-119 (+)] II; |
| | | unc-119(ed3) III; vsIsl3 IV; lin-15b(n765) X. |
| 28 | JPS146 | vxSi [Papl-1: apl-1: unc54UTR, Cbunc-119 (+)] II; |
| | SC_Papl-1: apl-1 | unc-119(ed3) III. |
| 29 | JPS150 | vxSi128 II; unc-119(ed3) III; vsIs13 IV; egl-1(ok1418) V; |
| | | lin15b(n765) X. |
| 30 | JPS151 | vxEx151[Prab3::mCherry]; vxSi1 II; unc-119(ed3) III; |
| | Prab3::mCh | vsIs13 IV; lin-15b(n765) X. |
| 31 | JPS152 | vxEx151[Prab3::mCherry]; vxSi38 II; unc-119(ed3) III; |
| | | vsIs13 IV; lin-15b(n765) X. |
| 32 | JPS166 | VxSi1 II, juIOs76 II; unc-71(ju156), unc-119(ed3) III. |
| 33 | JPS167 | vxSi1 II; unc-119(ed3) III; ced-3(ok734), vsIs13 IV; |
| | | lin-15B (n765) X. |
| 34 | JPS175 | vxSi175[Prab3::huAPPC59::unc-54UTR, Cb-unc-119 (+)] II; |
| | SC_APPC59 | unc-119(ed3) III; vsIs13 IV; lin-15b(n765) X. |
| 35 | JPS176 | vxSi176[Prab3::huAPPN636::unc-54UTR, Cb-unc-119 (+)] II; |
| | SC_APPN636 | unc-119(ed3) III. |
| 36 | JPS178 | vxSi178[Prab3::mCherry::unc-54UTR, Cb-unc-119 (+)] II; |
| | SC_mCh | unc-119(ed3) III. |
| 37 | JPS188 | vxEx188[Papl-1::apl-1]; vxSi1 II; unc-119(ed3) III; |
| | Papl-1::apl-1 | vsIs13 IV; lin-15b(n765) X. |
| 38 | LX929 | vsIs48[unc-17::GFP] |
| 39 | LX959 | vsIs13 IV; lin-15B(n765) X. |
| 40 | MT2236 | egl-1(n4065) |
| 41 | RB1021 | crt-1(ok948) |
| 42 | RB1305 | egl-1(1418) |
| 43 | RB885 | ced-3(ok734) |
| 44 | FX01826 | ced-6(tm1826) |
| 45 | CZ1931 | juIs76 II; unc-71(ju156) III. |
| 46 | EG4322 | ttTi5605 II; unc-119(ed3) III. |
| 47 | EG5003 | unc-119(ed3) III; cxTi10882 IV. |

Transgene Construction.

Transgenic animals with a single copy of pan-neuronal apl-1,huAPP695 full length, huAPP695N363 extracellular/transmembrane region, or huAPP695C59 intracellular region were generated through the MOSSCI technique as previously described in Frokjaer-Jensen C., Davis M. W., Hopkins C. E., Newman B. J., Thummel J. M., Olesen S. P., Grunnet M., and Jorgensen E. M. (2008) Single-copy insertion of transgenes in Caenorhabditis elegans. Nat Genet. 40, 1375-83. Briefly, we used Gateway technology (Invitrogen) to construct a vector with the apl-1 (genomic) or huAPP695(cDNA) gene driven by the pan-neuronal promoter (Prab-3) and an unc-54 UTR adjacent to the Cb_unc-119(+) positive selection marker. Transgenes were flanked by Mos1 transposon insertion sites complementary to a specific region on the 2nd(ttTi5605) or 4th(cxTi10882) chromosome. unc-119 mutant worms with specific Mos1 insertions sites were then injected with DNA. Selection of mobile, non-fluorescent progeny led us to identify single-copy insertion animals. Full insertion of the apl-1 or APP was confirmed by PCR and sequencing across the full transposon insertion site. Strains were then termed SC_apl-1,SC_APP, and SC_APPC59 to signify a single gene copy insertion. A control strain (designated WT for this paper) was also generated that contained only a single inserted copy of the Cb_unc-119(+) positive selection marker. Transgenic animals with SC_apl-1 or SC_APP genes tagged with mCherry were generated as above with the mCherry cDNA sequence fused to the unc-54 UTR to tag the C-terminal of the protein product. An additional strain using MOSSCI to integrate a single copy of mCherry cDNA driven by Prab-3 with unc-54 3′ UTR was also generated (SC_mCh). The sequence of the inserted mCherry gene was confirmed by sequencing of genomic DNA. Transgenic animals with multiple copies of pan-neuronal apl-1 or APP were also generated through injection (MC_APP and MC_apl-1 respectively). These multi-copy strains displayed a high level of lethality and developmental defects (Supplementary FIG. 26B). DNA (25-30 ng) vectors described above were injected into unc-119 mutant animals with fluorescent markers only. Formation of an extra-chromosomal array containing multiple gene copies was confirmed in mobile progeny that retained fluorescent markers. Expression level of sequence common to both APP and apl-1 genes was determined using RT-PCR with primers forward AAGCAGTGCAAGACCCAT (SEQ ID NO. 1) and reverse TCATCATCGTCCTCATCATCA (SEQ ID NO. 2).

Quantification of Percent Neurodegeneration.

All analyses were completed with the experimenter blind to genotype and drug treatment. Age-synchronized animals were immobilized on 2% agar pads containing 0.7 mM sodium azide. Neurons that had broken/missing axonal projections, dimly-lit somas with missing projections or absent GFP labeling in the appropriate neuronal location were classified as degenerating. Serotonin neurons included NSM, ADF, AIM, RIH, HSN class members. All animals were evaluated within 10 minutes of azide treatment. In cases where the worm strains were too defective in egg laying to reach day 3 or 5 adult stage, animals were treated at L4-larval stage onward with the sterilization drug 5-fluoro-2′-deoxyuridne (0.12 mM final) (Sigma). We found that this drug had no effect on the progression of degeneration of VC neurons (e.g. compare untreated animals in FIG. 20D to drug-treated animals in FIG. 22A). The percentage of VC4 and VC5 neurons that succumbed to APP-induced degeneration was compared for different groups (strain, age, drug condition) using planned non-parametric X² analyses.

Protein Localization Analysis.

Day-1, -3, and -5 adult mCherry-tagged APP and APL-1 strains were immobilized on 10% agarose pads containing microbeads. Animals expressing GFP/mCherry-tagged transgenes were observed under a Leica laser scanning confocal microscope (Leica TCS SP5 II) with a 63× oil-immersion objective (numerical aperture: 1.4). When two channels were used, images are acquired sequentially with the pinhole diameter set to 1.2 Airy units. Z-stacks were then taken at 40× magnification under red and green fluorescence separately, then pooled together to visualize co-localization with ImagePro Plus (Mediacybernetics). For comparison of fluorescence intensities in different areas of the worm, images were acquired under an identical exposure time, gain and pinhole diameter.

Laser Ablation of Neurons.

Neurons were ablated and VC neurons were visualized using an integrated fluorescent strain, in which VC neurons are labeled with GFP (strain LX959). All ablations were compared with shams of the same genetic background.

Pharmacology.

Drugs were freshly prepared in buffer (M9) solution and pipetted onto small seeded plates. These plates were allowed to dry at least 2 hours at RT before use. The final drug concentrations of solid media are: Dimebon, 50 μM (Tocris); P7C3, 50 μM. Well-fed larval stage 4 animals were picked onto seeded plates with or without drug. Animals were then allowed to age at 20° C. for ˜20 (day 1 adult), ˜68 (day 3 adult) and ˜116 hours (day 5 adult) before analysis. Controls using buffer with no drug were performed on each strain for each assay, and each assay was repeated multiple (3-8) times. Data were then averaged and statistically compared with planned non-parametric X2 analyses (Zar, 1999).

Percent Lethality.

The number of eggs laid by 10 first-day adult worms on bacterial plates (0P50) over 3 hours was counted, and surviving adult-stage progeny were then counted 72 hours later. Assays were repeated at least 3 times and percentages that failed to survive to adulthood were averaged.

Egg-Retention Assay.

Plates of non-starved adult worms were bleached to yield a synchronized population of eggs. Eggs were allowed to develop for ˜55 hours (day 1 adult) and ˜103 (day 3 adult) hours at 20° C. Animals were then individually dissolved in 1N NaOH, and eggs retained within the dissolved adult animal were counted.

Swimming Kinematics.

Worms were submerged in NGM liquid and recorded for 1 minute following a 2-minute acclimation period. Spine analysis software was then used to calculate and plot the midbody angle of the worm as described in Pierce-Shimomura J T, Chen B L, Mun J J, Ho R, Sarkis R, McIntire S L. 2008. Genetic analysis of crawling and swimming locomotory patterns in C. elegans. Proc Natl Acad Sci USA. 105: 20982-7, incorporated by reference. Head-bend frequency was determined based on the average time it took an animal to make 20 complete-cycle head bends. Defective swimming of single-copy APP or apl-1 strains could not be simply explained by accumulated eggs restricting motion because animals sterilized with 5-fluoro-2′-deoxyuridne (0.12 mM final) (Sigma) displayed the same defect.

Results

Pan-Neuronal Overexpression of APP Induces Age-Related Degeneration of a Specific Subset of Cholinergic Neurons

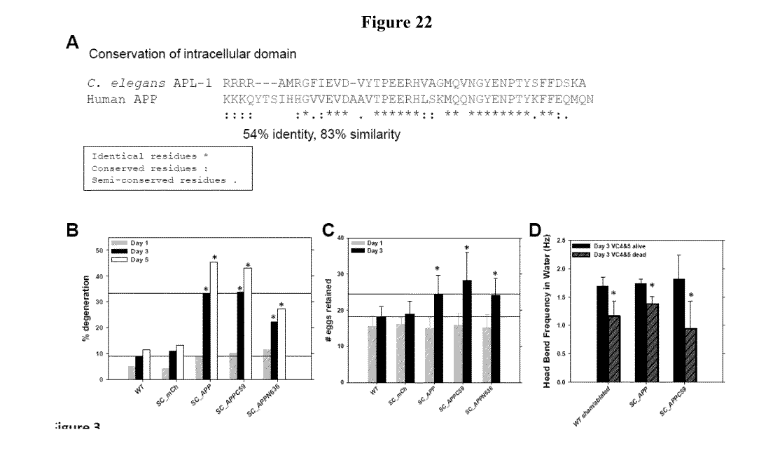

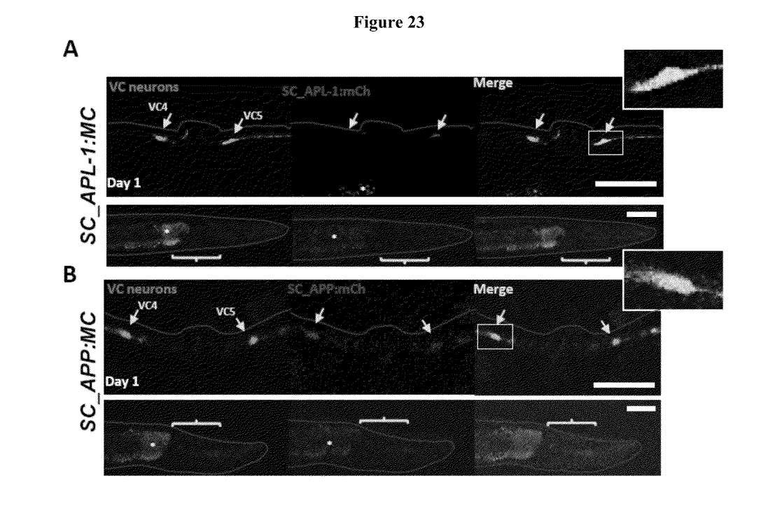

Both human APP and nematode apl-1 are widely expressed throughout the nervous system as well as in many other non-neuronal tissues. To focus on the nervous system, we generated strains that used a pan-neuronal promoter (Prab-3) to express a single wild-type copy of human neuronal APP, or an extra single copy of apl-1, knocked into a specific locus on the second chromosome. These transgenic strains are designated SC_APP and SC_apl-1 strains respectively. Both strains appeared normal in morphology and development from egg to young adult, and displayed an expected 2-fold higher expression of sequence common to both genes compared to wild type (FIG. 26A). We noticed, however, that SC_APP and SC_apl-1 worms displayed an abnormal hinging motion at the midbody during swimming on the third day of adulthood. This corresponds to post-reproductive peak and thus is considered “middle age” for C. elegans (FIG. 20A). Coincident with this phenotype, we observed the degeneration of certain neurons apparent as one or more vacuoles at the midbody (FIG. 21A, inset). The health of neurons with the aid of different fluorescent reporters was also investigated.

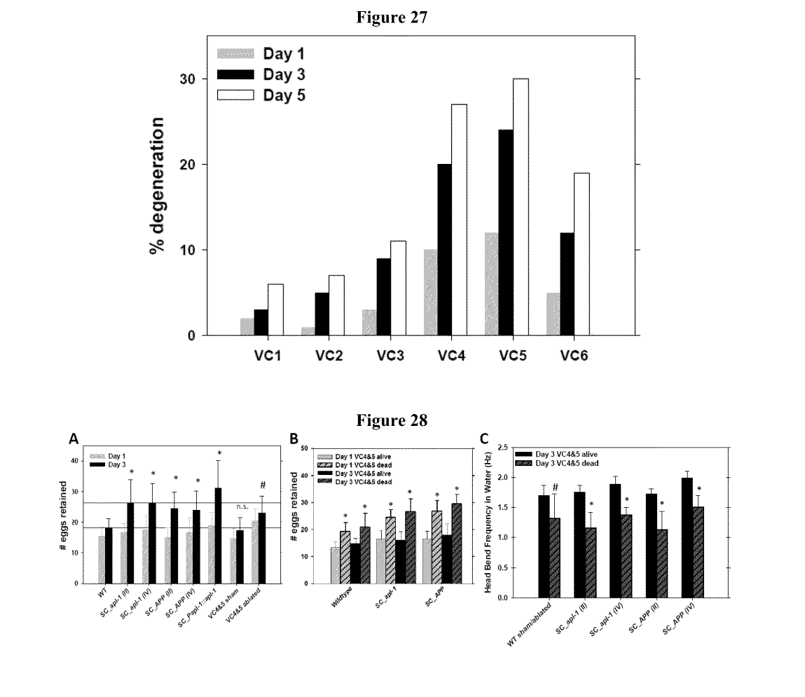

We discovered that the VC-class cholinergic (ACh) neurons were often absent or dying in the SC_APP and SC_apl-1 strains in middle-aged adults. Out of the six VC neurons, the VC4 and VC5 neurons (VC4&5) degenerated most reliably (FIG. 27; FIG. 20B,C). Thus, for convenience, we confined our further analyses to these two cholinergic neurons. Compared to a control “wild-type” strain (see methods), our AD model strains had a small but significant percentage (−10%) of VC4&5 neurons that degenerated by the first-day of adulthood, and 30-40% of VC4&5 neurons that degenerated by the third day of adulthood (FIG. 20D). Other classes of neurons, including those that are adjacent to the VC neurons and throughout the ventral nerve cord appeared healthy in appearance in our AD model strains (FIG. 20D). These included all 25 GABAergic neurons in the worm nervous system, as well as the 24 VA- and VB-class cholinergic neurons in the nerve cord. Likewise, nine serotonergic (5HT) neurons appeared normal in our AD models (FIG. 20D). Thus, after direct inspection of 64 of the 302 neurons that compose the C. elegans nervous system (White et al., 1986), we find that the six VC-class cholinergic neurons are particularly vulnerable to APP-induced degeneration.

Human AD is associated with an age-related decline in memory and cognition that depends on proper function of a vulnerable subset of cholinergic neurons. We found that our AD models displayed a similar decline in behaviors with age. We focused on two behaviors: egg laying and swimming, which depend, in part, on the VC neurons and many other neurons. SC_APP and SC_apl-1 strains retained a normal number of eggs in first-day adults, but retained significantly more eggs than wild-type and a control strain (designated WT herein, see methods) in third-day adults (yellow arrows in FIG. 21A,B; FIG. 28A). Likewise, our AD models exhibited defective swimming in third-day but not first-day adults. Frequencies of head-bending were slightly decreased (FIG. 28C) and bends at the midbody were even slower and biased towards the ventral side (FIG. 21C). The pattern of degeneration and behavioral defects could be not explained by a non-specific effect of the chromosomal integration site because integration of these genes into a different locus yielded identical results (FIG. 26C; FIG. 22A,C). Nor could it be explained by the particular Prab-3 pan-neuronal promoter because we also observed the same pattern of degeneration and behavioral defects when using the endogenous apl-1 promoter that expresses throughout the nervous system and additional tissues (FIG. 26C; 28A,C).

Further experiments with laser ablation revealed that the age-related decline in behaviors could be primarily explained by the death of the VC4 and VC5 cholinergic neurons. Ablation of VC4&5 in a WT background caused retention of a modest but significant number of eggs in first-day adults, and even more eggs in third-day adults (FIG. 21B; FIG. 28A). Another striking effect of APP and apl-1 overexpression is ventral-hinged swimming where over 80% of the single swim-cycle lags on the ventral side. Ablation of VC4&5 neurons recapitulated this unique phenotype and decreased head-bend frequency during swimming (FIG. 21C; FIG. 28C). Behavioral defects appeared to be due to the death of VC neurons rather than their dysfunction because there was a perfect correlation between individuals with behavioral defects and those with degeneration of VC4&5 neurons in WT and AD model strains (FIG. 28B,C).

Distinct Portions of APP Induce Patterned Neurodegeneration

The C. elegans APL-1 protein is highly homologous to human APP in several regions, especially in the intracellular region (FIG. 22A). The remaining region with transmembrane and extracellular portions is less well conserved, and notably lacks homology with Aβ. This suggested to us that the intracellular region may be more important to produce neurodegeneration in C. elegans. We tested if pan-neuronal expression of a single copy of only the conserved intracellular portion of APP(C59) produced neurodegeneration with a new transgenic strain SC_APPC59. We found that it was sufficient to recapitulate the same pattern of degeneration and behavioral defects observed in the SC_APP strain that expressed full-length human APP (FIG. 22B-D). Therefore, overexpression of the conserved intracellular portion is sufficient for age-related patterned neurodegeneration in C. elegans. We next tested whether a single copy of the remaining combined extracellular and transmembrane portion (N636) produced degeneration with an additional transgenic strain SC_APPN636. This strain also showed degeneration and associated VC4&5-dependent behavioral defects (FIG. 22B,C). Together, these additional transgenic strains demonstrate that distinct portions of wild-type human APP protein can lead to degeneration in C. elegans just as they appear to in different rodent models.

Pan-Neuronally Overexpressed APP Accumulates in Select Cholinergic Neurons Preceding Degeneration in Middle Age

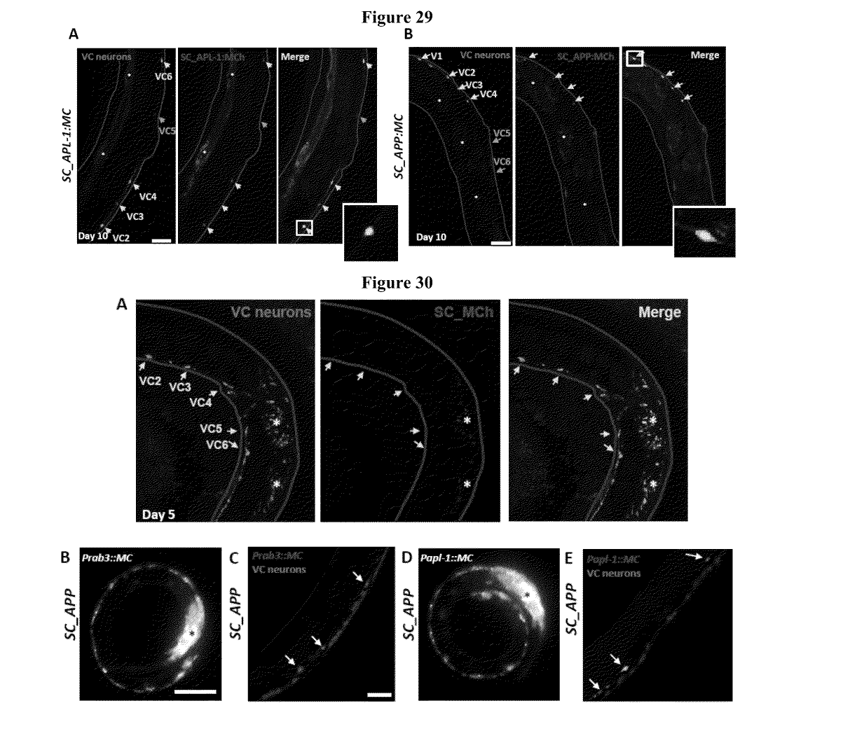

The widespread expression of APP in the nervous system of humans belies the selective vulnerability of specific cholinergic neurons in AD. Similarly, it was unclear why pan-neuronal expression of APP or apl-1 would lead to selective degeneration of the VC cholinergic neurons. To visualize patterns of APP expression in C. elegans, we constructed new transgenic strains with a single copy of C-terminal mCherry-tagged APP or APL-1 (strains SC_APP:mCh and SC_apl-1:mCh). Despite being expressed by a standard pan-neuronal promoter (Prab-3), just as in the SC_APP and SC_apl-1 strains above, we only detected mCherry fluorescence in the six VC-class cholinergic neurons with confocal microscopy (FIG. 23). The mCherry signal became apparent in first-day adults, and increased up until individual VC neurons died. In rare individuals that reached advanced age, mCherry expression was visible in all VC neurons, but not elsewhere (FIG. 29).

We next generated a series of transgenic strains as controls to test whether accumulation could be due to a number of unexpected scenarios. One possibility is that the mCherry protein itself tends to accumulate in VC neurons. To test this idea, we generated a strain that expressed a single copy of mCherry with the same pan-neuronal promoter (strain designated SC_mCh). As expected, however, we failed to detect any fluorescence with such a low gene dose (FIG. 30A). In addition, the specificity of mCherry-tagged protein accumulation in VC neurons could not be attributed to an artifact of the Prab-3 promoter because we confirmed that a Prab-3::mCherry transgene expressed throughout the nervous system from embryonic development onward in WT and our AD model backgrounds (FIG. 30B,C). Alternatively, VC neurons might accumulate APP and APL-1 if these neurons did not normally express apl-1. However, we found that the endogenous apl-1 promoter could drive expression of mCherry throughout the nervous system, as previously reported, including in the VC neurons in WT and our AD model backgrounds (FIG. 30D,E). Thus, we conclude that the selective degeneration of VC cholinergic neurons in C. elegans is likely caused by the selective accumulation of APP or APL-1.

Apoptotic Signaling is Required for APP-Induced Neurodegeneration

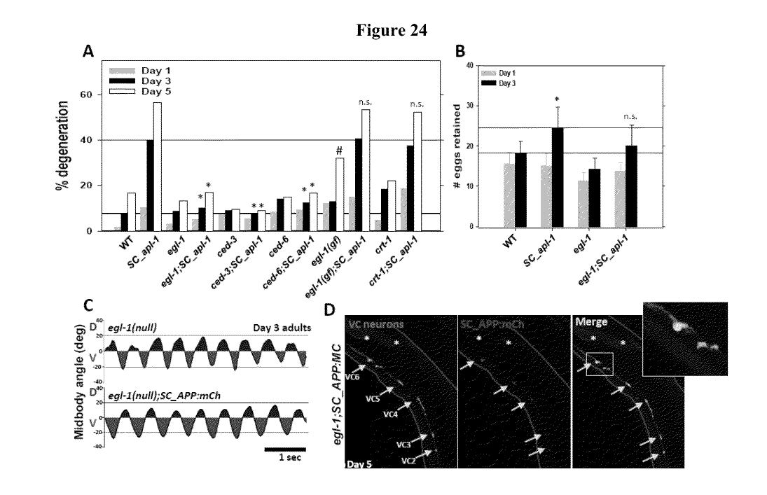

Many highly conserved components of necrotic, apoptotic, and phagocytic pathways that underlie cell death have been discovered in C. elegans. Which ones are essential for APP-induced neurodegeneration? First, we ruled out a role for conventional necrosis because elimination of crt-1, the single homolog of the essential necrotic gene calreticulin, had no effect on degeneration (FIG. 24A). Next, we found an essential role for apoptosis with several experiments. Deletion of egl-1, a homolog of BH3 (Bcl-2 homology region 3) type activators of apoptosis in mammals, prevented degeneration (FIG. 24A). Additionally, deletion of egl-1 partially rescued behavioral defects in SC_APP and SC_APL-1 strains (FIG. 24B,C). Conversely, activating apoptotic signaling with a gain-of-function mutation in egl-1 did not exacerbate the incidence of degeneration (FIG. 24A). Indeed, the egl-1(e) mutant showed age-related degeneration even without APP (or apl-1) overexpression. Consistent with an essential role for apoptosis, deletion of ced-3, a homolog of the mammalian apoptotic caspase-9/ICE, similarly prevented degeneration (FIG. 24A). Likewise, elimination of ced-6, the homolog of mammalian phagocytotic gene GULP, prevented degeneration and preserved function of VC4&5 neurons (FIG. 24A). This suggests that in our AD models CED-6 has a primary apoptotic role to act as a “killer” signal as found in other scenarios. Our results demonstrate that APP-induced degeneration requires members of the conserved apoptotic pathway including EGL-1, CED-3, and CED-6.

If abnormal accumulation APP (or APL-1) causes degeneration, might interference of apoptotic signaling prevent accumulation of APP (or APL-1)? Instead, we found that the level and pattern of mCherry-tagged APP (or APL-1) expression in an egl-1(null) background was indistinguishable from that of SC_APP:mCh and SC_APL-1:mCh strains (FIG. 24D). Although we cannot rule out the possibility that loss of egl-1 causes subtle redistribution of mCherry-tagged protein at the subcellular level, our results strongly suggest that defects in apoptotic genes prevent degeneration by blocking APP-triggered entrance into the apoptotic pathway.

P7C3 Prevents APP-Induced Neurodegeneration while Maintaining Neural Function

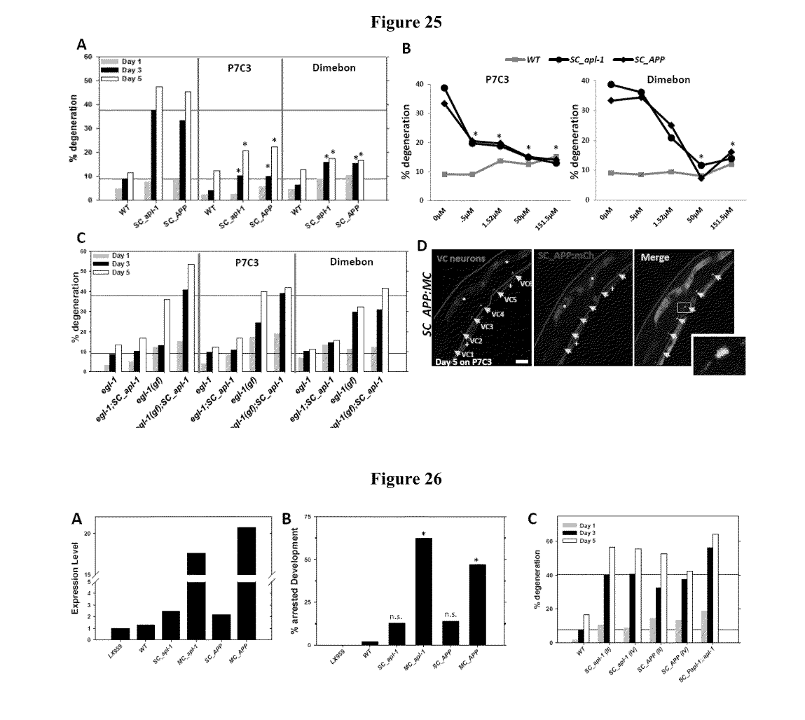

We next tested whether the new putative neuroprotetive compound P7C3 could also prevent APP-induced degeneration. P7C3 was recently discovered in an unbiased screen for small molecules that increase the number of adult-born neurons in the hippocampus of mice potentially by increasing their survival (preventing their death). The mechanistic basis for the neuroprotective effects of P7C3 remains unknown. Animals were treated with P7C3 (50 μM) from L4-larval stage onward (FIG. 20A). P7C3 treatment significantly prevented neurodegeneration induced by APP or apl-1 (FIG. 25A). The structure of P7C3 resembles Dimebon, a potential drug for AD. We found that both drugs prevented neurodegeneration (FIG. 25A). A dose response analysis found that P7C3 was two-orders of magnitude more potent than Dimebon (FIG. 25B).

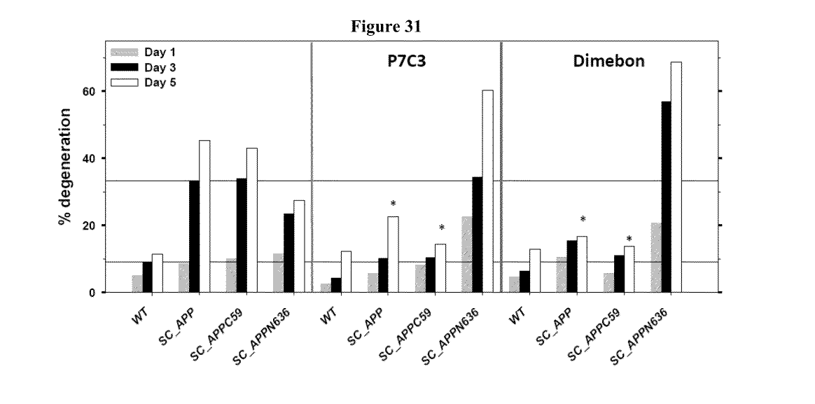

To determine whether P7C3 and Dimebon could prevent degeneration elicited by distinct portions of APP, we assayed the protective effects of each drug on our strains that overexpressed different portions of APP. Surprisingly, we found both drugs prevented degeneration for the SC_APPC59 strain, but failed to provide protection for the SC_APPN636 strain (FIG. 31). Protective effects against degeneration for the SC_APPC59 strain extended to protection of egg-laying and swimming behaviors that depend on the VC4&5 neurons (data not shown). These results suggest that when separated, different portions of human APP molecule may initiate apoptotic degeneration via different entrances, only one of which might be protected by P7C3 and Dimebon.

Lastly, we used mCherry-tagged transgenes to determine whether the drugs might prevent degeneration by stopping accumulation of APP or APL-1. While drug-treated SC_APP:mCh or SC_apl-PmCh individuals advanced in age without behavioral deficits (swimming and egg laying), mCherry-tagged protein accumulated specifically in the VC cholinergic neurons (FIG. 25D). To narrow down the point at which these drugs act in the neurodegenerative pathway we tested how our AD models responded with different apoptotic mutations. P7C3 and Dimebon offered no further protection from degeneration in an egl-1 (null) mutant background (FIG. 25C). Conversely, these drugs failed to prevent degeneration in SC_APP and SC_apl-1 strains with a gain-of-function mutation in egl-1 (FIG. 6C). Taken together, these results suggest that the drugs act upstream of EGL-1 apoptotic signaling. Thus, P7C3 and Dimebon appear to prevent degeneration by blocking accumulated APP from triggering apoptotic signaling.

Drugs may easily be tested for in vivo neuroprotective effects with C. elegans. In less than one week, protective effects can be accessed by direct visualization of fluorescently labeled cholinergic neurons. Moreover, the functional integrity of these specific neurons can be assessed with simple behavioral assays. In contrast to most AD drugs in clinical trials that aim to reduce accumulation of APP and plaques, we show that P7C3 represents a novel drug class because it can prevent apoptotic degeneration and preserve neuronal function even in the face of APP accumulation. Because P7C3 and Dimebon show favorable pharmacological profiles in mice and humans respectively, our results validate the use of C. elegans for the evaluation of potentially beneficial compounds in the treatment of AD and other neurodegenerative disorders with unprecedented speed and cost effectiveness.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A genetically modified nematode belonging to genus Caenorhabditis comprising a single additional copy of a gene that encodes an ortholog of a gene associated with a neurodegenerative disease.

2. A genetically modified nematode belonging to genus Caenorhabditis comprising a single copy of at least a portion of a human gene associated with a neurodegenerative disease.

3. The genetically modified nematode of claim 1, wherein the gene is apl-1, ptl-1, or Y110A2Al.13.

4. The genetically modified nematode of claim 2, wherein the gene is APP, TAU, or PIN1.

5. The genetically modified nematode of claim 1, wherein the gene is pink-1, K04F10.1, atx-2, atx-3, abtm-1, unc-23, irk-1, irk-2, or ZK1320.13.

6. The genetically modified nematode of claim 2, wherein the gene is PINK, SCA1, ataxin-2, ataxin-3, ABCB7, SYNJ, 11RK3, or PCP4.

7. The genetically modified nematode of claims 1-6, wherein the gene comprises a tag.

8. The genetically modified nematode of claims 1-2, wherein said nematode belongs to the species selected from the group consisting of: elegans, vulgaris, and briggsae.

9. The genetically modified nematode of claims 1-2, wherein said nematode is Caenorhabditis elegans.

10. The genetically modified nematode of claims 1-2, further comprising a phenotype associated with the expression of the gene, the phenotype selected from the group consisting of: a defect in egg laying; a defect in swimming; a defect in the formation of the vulva; plaque-like deposits in the nervous system; a retarded development; a decreased number of descendants; fluorescence.

11. A genetically modified nematode belonging to genus Caenorhabditis comprising a single additional copy of one or more of apl-1, ptl-1, and Y110A2A1.13.

12. A genetically modified nematode belonging to genus Caenorhabditis comprising a single copy of human APP, TAU, or PIN1.

13. A composition comprising genetically modified nematodes according to claims 1-2.

14. A genetically modified nematode belonging to genus Caenorhabditis comprising one or more of the following genes under the control of the egl-1 promoter: akt-1, gld-1, ape-1, abl-1, fsn-1, egl-1, egl-1, ced-4, ced-3, ced-1 and ced-6.

15. A method comprising:
exposing a genetically modified nematode according to claim 1-2, or 14 to a substance; and
observing a phenotypic change in the nematode.

16. The method of claim 15, wherein the step of a phenotypic change in the nematode is automated.

17. A method comprising:
exposing a genetically modified nematode according to claim 1-2, or 14 to a substance;
observing the effect of the substance on a phenotype of the nematode; and
comparing the effect on the observed phenotype in the presence of the substance to the observed phenotype in the absence of the substance, wherein the substance is identified as a pharmaceutical for the treatment or prevention of a neurodegenerative disease.

18. The method of claim 17, wherein said substance attenuates or abolishes the observed phenotype.

19. Method of claim 17, further comprising exposing a second substance and observing the effect of the second substance on the observed phenotype.


SEQUENCE LISTING (2 sequences)

SEQ ID NO: 1 - DNA, 18 bp, Artificial Sequence
  Primer
aagcagtgcaagacccat

SEQ ID NO: 2 - DNA, 21 bp, Artificial Sequence
  Primer
tcatcatcgtcctcatcatca